(19) United States
(12) Patent Application Publication
(10) Pub. No.: US 2022/0226500 A1
(43) Pub. Date: Jul. 21, 2022

(54) FULLERENIC ELLAGIC LUTEOLIN AND METHODS

(71) Applicant: Sinapu LLC, Monrovia, CA (US)

(72) Inventor: Peter Butzloff, Walnut, CA (US)

(21) Appl. No.: 17/591,126

(22) Filed: Feb. 2, 2022

Related U.S. Application Data
(63) Continuation of application No. PCT/US22/12369, filed on Jan. 13, 2022.
(60) Provisional application No. 63/139,721, filed on Jan. 20, 2021.

(51) Int. Cl. A61K 47/69; A61K 47/54; A61K 31/366; A61K 31/352; A61K 9/16

(52) U.S. Cl. CPC A61K 47/6949; A61K 47/54; A61K 9/1664; A61K 31/352; A61K 47/548; A61K 31/366

(57) ABSTRACT

A nanoparticle composition of buckminsterfullerene with ellagic acid is provided that becomes substantially more efficacious to maintain or re-establish benign healthy cellular homeostasis with the functionalization of at least about 10% luteolin. This composition is formulated to prevent or to treat chronic obstructive pulmonary disorder (COPD). In addition, the ability to penetrate hydrophobic malignant tissues via desulfurization is promoted with the addition of phosphonate pendant groups. This further enables the composition to penetrate fungal spores, the hydrophobic regions of uncontrolled cellular proliferation, neoplasms, degenerative malignancy, and to help treat chronic inflammatory diseases associated with or leading to induce cancer in susceptible cells. The composition can be produced at low temperatures through reactive shear milling. Delivery methods include ingestion, topical application, topical buccal application, inhalation, or injection when used as a medicament or as a food supplement.

### CROSS-REFERENCE TO RELATED APPLICATION

This application is a continuation of International Application PCT/US22/12369 filed on Jan. 13, 2022 which claims the benefit of U.S. provisional patent application 63/139,721 filed on Jan. 20, 2021, both of which are incorporated herein by reference in their entireties.

### BACKGROUND

### 1. Field of Invention

The present invention is a composition of buckminsterfullerene with ellagic acid, luteolin, and disodium phosphonate pendant functional groups in selected combinations, with methods of use to prevent or to treat chronic respiratory illness such as obstructive pulmonary disorder (COPD), and to help treat uncontrolled cellular proliferation, neoplasms, degenerative malignancy, and chronic inflammatory diseases associated with cancers or associated with a tendency to induce cancer in susceptible cells. These same properties promote usage for anti-aging in skin and to promote preventative health for topical buccal tissues. Delivery methods include ingestion, topical application, inhalation, or injection when used as a medicament or as a food supplement to maintain or re-establish benign healthy cellular homeostasis.

### 2. Background Art

It is estimated that 80% or more of the variation in cancer risk among tissues is caused by environmental factors. These factors result in genetic alterations that can lead to unregulated malignant cell proliferation, or cancer. These alterations create a cell metabolism imbalance, resulting in a build-up of undigested proteins, misfolded proteins, and inflammatory molecular fragments. Death to the individual may arise from a shift to poorly controlled catabolism (protein breakdown) of healthy cells. Part of this process may occur by usurpation of critical metabolic resources by the uncontrolled proliferation of tumorous or malignant cells that metastasize into cancer. Anabolism is the buildup of healthy cellular tissues from sugars and amino acid raw materials. One aspect of a cancer avoidance strategy is to help reassert a proper balance between catabolism and anabolism. Therefore, there has been a search for a preventative regimen or treatment for cancer that is able to correct the faulty reprogramming of the cell energy metabolism. The following factors have been found to contribute to the progression of cellular metabolic dysfunction: changes to the redox process, sustained antiproliferative signaling, evasion of growth suppressors, invasion (metastasis activation), enhanced replicative immortality, angiogenesis, and resistance to cell death.

The consensus among scientists and medical practitioners is that oxidative stress plays a major role in the development of cellular metabolic disease and cancer. Some of the current therapeutic strategies have put emphasis on the design of multiple functional properties into molecules or particles that enable them to target multiple enzymes or receptors associated with the development of cellular metabolic disease, allowing them all to operate at the same time to avoid or correct the dysfunctions leading to cancer. Of these, several natural antioxidants have been known for their ability to trap and scavenge free radical species, being bioflavonoid and polyphenolic components of plants with well understood antioxidant, antimutagenic, anticarcinogenic, and antihyperglycemic activities while having a cardioprotective effect. These properties arise from the ability to protect against especially reactive oxygen species (ROS) such as hydroxyl and nitrate free radicals. ROS are associated with causing damage to essential cellular components. One way that ellagic acid achieves this effect, is to decrease cellular oxygen consumption, which provides metabolic regulation.

Several studies indicate that ellagic acid polyphenol confers beneficial effects against several chronic pathologies associated with oxidative stress damage to cells, including multiple types of cancer, cardiovascular disease, and neurodegenerative diseases. In addition, ellagic acid provides significant antibacterial and antiviral properties that find use in wound healing.

Of these well-known effects, the in-vitro and murine studies of the anticancer properties of ellagic acid have attained great interest by demonstrating cancer cell proliferation inhibition. Malignant cell proliferation reduction was achieved by significantly reducing adenosine triphosphate levels while decreasing the electrical potential present at the inner mitochondrial membrane. Ellagic acid was also shown to activate beneficial adenosine monophosphate protein kinases (AMPK) and to reduce the hypoxia-inducible factor 1-alpha (HIF-1A) in cancer cells. The dysregulation and overexpression of HIF1A has been strongly evidenced in the current understanding of cancer biology, as well in angiogenesis, energy metabolism, cell survival, and tumor invasiveness. However, these effects are not able to be developed at present into any significant medicament and have not been demonstrated to confer significant protective effects in the human diet.

The present trend in the development of improved pharmaceutical and nutraceuticals is to create new agents that can join the best observed antioxidant effects with some design of a pharmacological molecule that can demonstrate control over cell signaling. However, at least in the case of ellagic acid, the reduction to practice in either of these fields has not been as successful as envisioned.

One significant limitation to the use of ellagic acid to improve health, is its poo stability of under physiological conditions. This explains the poor bioavailability and transport through the body to the targeted tissues requiring prophylactic protection or antiproliferative treatments. Ellagic acid has a solubility of 0.0097 grams per liter of water, making it very difficult to dissolve for treatment purposes. Moreover, the human body acts to significantly break down more than half of the absorbed blood plasma level of ellagic acid within one hour. However, even before reaching the blood plasma, ellagic acid consumed in oral form is significantly metabolized by the gastrointestinal microbiota to remove the pendant hydroxyl groups one at a time, to form a series of three urolithins.

While there is great promise in the beneficial effects of ellagic acid, there is a need to improve the known strategies to enhance its poor oral bioavailability. Moreover, attempts to perform encapsulation within microspheres or lipids to be used as carriers, or attempts to perform a molecular dispersion in polymer matrices to be used as carriers, have failed to protect ellagic acid from oxidation in the digestive process to allow a maximum benefit.

Yet another significant limitation to the use of ellagic acid, is in the lack of maturity of targeting this substance to dysfunctional cells by means of appropriate cell signaling. Such design failures in application to ellagic acid, are attributed to an incomplete understanding of cell signaling functions and protein messaging effects that are part of disease development. Cell signal interactions begin with surface charges at membranes. Surface charges are in contact with the cell cytosol, proteins, deoxyribonucleic acids (DNA), and the lipid membranes of the cell. Some signaling regions, such as the endoplasmic reticula of mitochondria, may become insufficiently engaged in reduction-oxidation that is associated with the development of tumors and cancers. This deficit, along with reactive oxygen species (ROS) associated in the aging process, are thought to contribute to dysfunction of the electron transfer cycle that allows proper cellular respiration to take place, and the result can be the production of misfolded proteins and runaway cell proliferation that are associated with many kinds of cancers and tumorous growths.

A delicate chemical balance of reduction and oxidation (REDOX) operates mitochondria and drives cellular function. Cellular health and benignity become compromised with genetically encoded and environmentally induced mis-development of anabolism and catabolism. The release of ROS by mitochondria is characterized by hydrogen peroxide (H₂O₂), and a wide variety of biological molecules involved with REDOX control. NADPH levels may become decreased, resulting in the reduced concentration of its precursor, nicotinamide adenine dinucleotide (NAD+). This leads to increased levels of the ratio of [NADP+]/[NADPH], leading to a cascade of pathological effects. These effects include increases in oxidative stress, mitochondrial electron transport chain dysfunction, and the promotion of inflammation in tissues.

It is well known and widely reported that ellagic acid polyphenols cause upregulated expression of the antiproliferative p53 protein. It is reported that ellagic acid can play an important role in the inhibition of well characterized standard cancer HepG2 cell proliferation in vitro. These phenomena may also be related to the decreasing nuclear factor kappa B (NF—B) activity, thereby activating the mitochondrial death pathway, which is associated with the loss of mitochondrial membrane potential, cytochrome C release, and caspase-3 activation. However, the effective transport and stability of ellagic acid polyphenols to target mitochondria in cancerous cells in-vivo remains problematic.

New and unique anti-inflammatory compositions targeting the well-known cellular roles of NADPH and superoxide dismutase (SOD) at the mitochondria have been or are continuing to be developed to treat diverse pathological cell conditions leading to inflammation and disease. Such conditions include but are not limited to cancer, cognitive decline, arthritis, diabetes, vascular disease, neurological disease, and colitis. Nowadays, multiple functions are designed into such substances to allow anti-Tumor Necrosis Factor Alpha (anti-TNFα), and anti-inflammatory interleukins such as anti-IL-6, and anti-IL-1 therapies simultaneously. Such multifunctional compositions are being tested in clinical trials for efficacy with a spectrum of outcomes. These substances are also being considered as interventions in the aging process to evaluate if any of the treatments might improve the health span of aging individuals. Unfortunately, many of these treatments or compositions are not bioavailable, being poorly soluble in water and being unable to pass cellular membranes. Typical drug loading of about 10% is achieved for nanoparticles of a narrow size distribution around an average size of about 100 nanometers when encapsulated in water soluble polymer micelles, suggesting complete dispersibility of such substances, but when doing so, this results in the substantial or complete masking of the therapeutic agent, along with poor targeting to cancer cells. The dual bioavailability and targeting problems remain significant obstacles to commercial and medical success of multifunctional antiproliferative prophylactic and therapeutic compositions.

Yet other treatments or compositions have therapeutic designs based on a philosophy of selectively poisoning malignant and dysfunctional cells by applying toxic chemicals, radiation, or both in some combination. This philosophy has met with limited success, but many cases are not solved, and outcomes can even be worsened by using this approach. The loss of life and severe economic impact to families and society from insufficiently comprehensive state-of-the-art treatments remains a burden that society can ill afford, even as some incremental progress is being made in this area.

What is therefore needed is a novel therapeutic strategy or unique material used to confer cellular protection and prevent, mitigate, or reverse cancerous pathology or illness arising from cellular dysfunction before irreversible or life-threatening damage progresses. Desirably, such a benignity treatment should include a prophylactic enhancement of DNA stability by removing sources of oxidation and free radical generation. It is believed the present invention provides such a composition, having a biological and electrochemical design to confer multiple therapeutic and prophylactic functions. The use of different carrier formulations enables appropriate methods of administration for this composition.

### SUMMARY OF THE INVENTION

This invention is a cluster of nanoparticles composed with carbon fullerenes covalently derivatized with phosphonates having oxidation state of three, and a polyphenol moiety being ellagic acid where this substance is pi-carbonyl bonded from at least one carbonyl group (C═O) to the aromatic regions of the fullerene phosphonate. The pendant acid phosphonates are neutralized with cations, preferably sodium, to form disodium phosphonate groups comprising a surfactant nature and having a viral protease inhibiting function via the phosphonate sulfurization reaction. This novel and unique fullerene composition possesses properties which reflect the singular free radical scavenging chemical function of fullerenes, the anti-proliferative function of acidic polyphenols, and the protease control function of cationic disodium phosphonates.

The result of these combined functions is to allow time for the proper re-integration of dysfunctional, senescent, or malignant cells by introducing an enhanced REDOX reversibility, and to directly inactivate reactive oxygen species (ROS) in mitochondria and at the surface membranes of cellular organelles to re-establish functional cellular homeostasis.

In one aspect, the function of the pi-carbonyl bonded polyphenolic functional groups is to control the reactive oxygen species that may cause protein and peptide polymerization in dysfunctional cells.

In a related aspect, the function of the pi-carbonyl bonds between ellagic acid, luteolin, and the core fullerene C60 provides a novel in-vivo stability design resistant to the digestive process.

In another aspect, the composition of ellagic acid and luteolin with fullerene penta-disodium phosphonate (FDSP) is to treat COPD using the method of inhalant delivery to the lungs and airways of a patient (person or animal) in need of treatment.

In a related aspect, the composition of FDSP-ellagic acid-luteolin is to treat valley fever, a type of fungal infection of the lungs and airways, using the method of inhalant delivery to a patient that is experiencing this type of chronic inflammatory bronchitis.

In another aspect, the function of the sodium phosphonates pendant from the fullerene is to control viral proteases as well as to inhibit or significantly reduce the pathological impact of malignant, cancer-causing viruses by means of the sulfurization reaction at the compositions containing phosphonate functional groups.

In another aspect, the surfactant properties of the phosphonate functional groups in C60-ellagic acid-luteolin-disodium phosphonate or C60-ellagic acid-disodium phosphonate is to uniquely empower the ellagic acid anti-cancer activity to reach into and diffuse throughout the very difficult to penetrate hydrophobic regions commonly expressed by tumor cells and cancer cells.

In another aspect, C60-ellagic acid-luteolin-disodium phosphonate promotes the functional lifetime of endogenous p53 protein in the human body, thereby allowing this protein to avoid becoming recycled and thereby allowing it to continue to perform ordinary DNA repair, in which this aspect leads to a greater resistance to future cancer.

In another aspect, the presence of the core fullerene molecule is to disassemble detrimental salt bridges between proteins that interfere with innate immunity response, by means of high negative charge density acquisition leading to the abstraction and sequestering of cations away from hydrophobic tumor proteins.

In a related aspect, the composition of the present invention directs mitochondrial signals arising from the high charge storage density of the core fullerene molecule. When combined with the pendant group functionality, the ability to meter out stored electrons or protons while simultaneously acting to quench free radicals serves to help regulate the process of oxidative phosphorylation. This regulation in turn modulates cellular homeostasis by self-adjusting the balance among cellular respiration, protein synthesis (anabolism), protein utilization and recycling (catabolism), while preventing mitochondrial membrane hyperpolarization.

In a related aspect, the fullerene chelation ability functions as a free radical recombination and detoxification center, thereby reducing inflammation and serving to boost innate immunity while reducing the tendency for autoimmune disease to inflict damage on tissues.

In another aspect, the FDSP ellagic acid luteolin composition is sequestered into the pores of food grade Transcarpathian zeolite (clinoptilolite) for the purpose of timed-release delivery of the orally administered composition.

In another aspect, the FDSP ellagic acid luteolin composition is heated to form a nano aerosol for the purpose of immediate aspirated delivery to the lungs, thereby providing access to the blood system for rapid release of the administered inhalant composition.

These and other advantages of the present invention will be further understood and appreciated by those skilled in the art by reference to the following written specifications, claims, and appended drawings.

Some embodiments are described in detail with reference to the related drawings. Additional embodiments, features, and/or advantages will become apparent from the ensuing description or may be learned by practicing the invention. In the drawings, which are not to scale, like numerals refer to like features throughout the description. The following description is not to be taken in a limiting sense but is made merely for describing the general principles of the invention.

### BRIEF DESCRIPTION OF THE DRAWINGS

Preferred embodiments of the invention will now be described, by way of example, with reference to the accompanying drawings, in which:

FIG. 1 illustrates the molecular structures of the raw materials that may be used in preselected combination to synthesize the present invention.

FIG. 2 illustrates two side views of the molecular structures of fullerene disodium phosphonates.

FIG. 3 illustrates one portion of a molecular structure of fullerene disodium phosphonate.

FIG. 4 illustrates a reaction between ellagic acid and/or luteolin with a C60 fullerene to produce pi-carbonyl bonded adducts.

FIG. 5 illustrates a reaction between the molecular structures of ellagic acid and/or luteolin with FDSP to produce pi-carbonyl bonded adducts.

FIG. 6 illustrates molecular structures with selected pi-bonded adducts, and desulfurization.

FIG. 7 illustrates the composition of the present invention disposed within the pores of Transcarpathian zeolite (clinoptilolite), and/or the pores of diatomaceous earth.

FIG. 8 is a flow chart showing exemplary steps to prepare a nano-aerosol fluid.

FIG. 9 is a flow chart showing exemplary steps to prepare oral formulations.

FIG. 10 is a flow chart showing exemplary steps to prepare topically administered FDSP-ellagic-luteolin for skin care and an exemplary method to create the buccally administered formula.

FIG. 11 illustrates an exemplary method of personal inhalant administration of a nano-aerosol formulation.

FIG. 12 illustrates exemplary methods of personal administration of the topical skin care formulation, and for the buccal formulation.

FIG. 13 illustrates experimental FTIR data for ellagic acid raw material.

FIG. 14 illustrates experimental FTIR data for luteolin raw material.

FIG. 15 illustrates experimental FTIR data for C60-ellagic acid-luteolin.

FIG. 16 illustrates experimental FTIR data for FDSP raw material.

FIG. 17 illustrates experimental FTIR data for FDSP ellagic acid luteolin.

FIG. 18 illustrates experimental negative mode MALDI-TOF mass spectrograph data for FDSP raw material.

FIG. 19 illustrates experimental negative mode MALDI-TOF mass spectrograph data for FDSP ellagic acid luteolin.

### DETAILED DESCRIPTION OF THE INVENTION

The following detailed description, taken in conjunction with the accompanying drawings, is merely exemplary in nature and is not intended to limit the described embodiments or the application and uses of the described embodiments. Any implementation described herein as “exemplary” or “illustrative” is not necessarily to be construed as preferred or advantageous over other implementations.

Furthermore, there is no intention to be bound by any expressed or implied theory presented in the preceding technical field, background, brief summary or the following detailed description. It is also understood that the specific devices, systems, methods, and processes illustrated in the attached drawings, and described in the following specification, are simply exemplary embodiments of the inventive concepts defined in the appended claims that there may be variations to the drawings, steps, methods, or processes, depicted therein without departing from the spirit of the invention. All these variations are within the scope of the present invention. Hence, specific structural and functional details disclosed in relation to the exemplary embodiments described herein are not to be interpreted as limiting, but merely as a representative basis for teaching one skilled in the art to variously employ the present embodiments in virtually any appropriate form, and it will be apparent to those skilled in the art that the present invention may be practiced without these specific details.

Various terms used in the following detailed description are provided and included for giving a perspective understanding of the function, operation, and use of the present invention, and such terms are not intended to limit the embodiments, scope, claims, or use of the present invention.

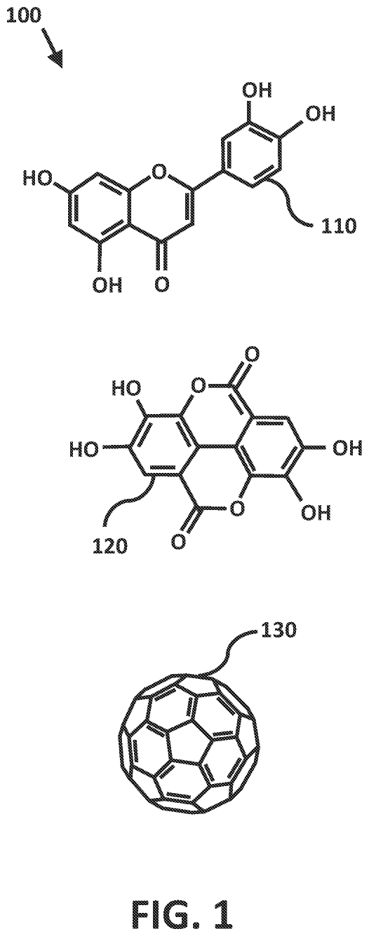

FIG. 1 illustrates three molecular structures 100 used in the present invention. The molecular structure of the bioflavonoid luteolin 110 has molecular formula C₁₅H₁₀O₆ and is commercially available as a concentrated plant extract or as a synthetic organic food grade food supplement powder. The polyphenol molecule ellagic acid 120 has molecular formula C₁₄H₆O₈ and is commercially available as a concentrated plant extract, often from pomegranate fruit, or as a synthetic organic food grade food supplement powder that is a solid at room temperature. Buckminsterfullerene 130 is a single molecule comprised of 60 carbon atoms arranged as a sphere and is frequently represented as C60. In various embodiments the C60 raw material that is used for synthesizing compositions of the present invention comprises 99.95% or better purity of vacuum sublimed C60. Substances 110, 120, 130 may be used to help create, process, or deliver parts of the composition of the present invention or their metabolites according to the teachings of the present invention.

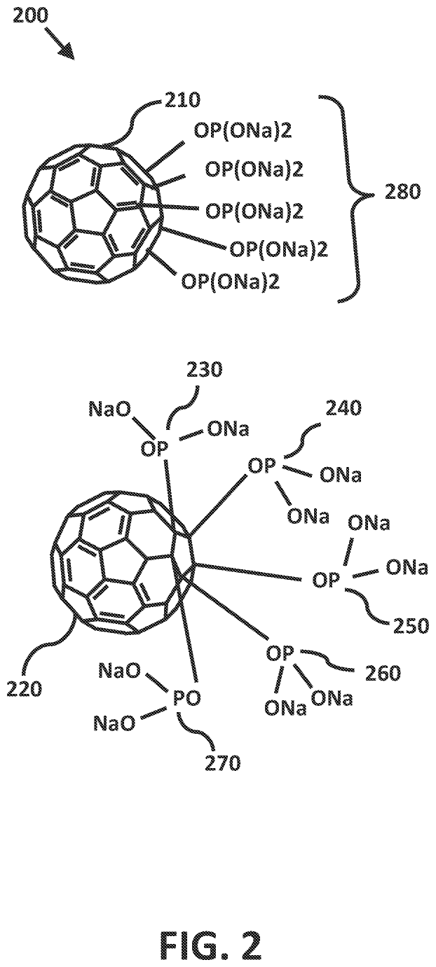

FIG. 2 illustrates two alternative side view molecular structures of FDSP, 200. One mole of C60 fullerene can be reacted with 5 moles of phosphonic acid having oxidation state of 3 to form one mole of FDSP, or this material may be purchased commercially as a pure raw material in powder form comprising a core C60 fullerene 210, 220 that is covalently bonded with five phosphonate groups as shown in 230, 240, 250, 260, 270 or as shown collectively in the bracketed region 280. FDSP 200 is commercially available as a raw material to be used as an ingredient to help create, process, or deliver parts of the composition of the present invention or their metabolites according to the teachings of the present invention.

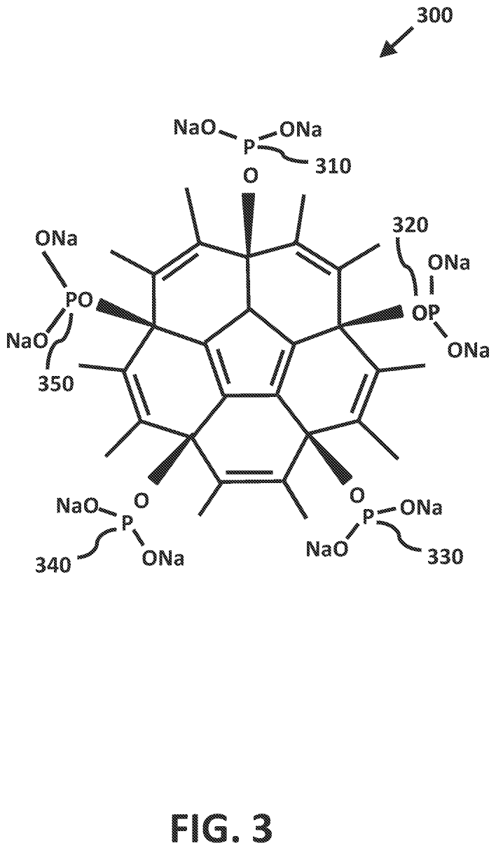

FIG. 3 illustrates a partial section of a front view of the FDSP molecular structure 300 to show a flat perspective of the five covalently bonded disodium phosphonate groups 310, 320, 330, 340 350 that are symmetrically deposed with respect to one central pentagonal carbon ring having delocalized and highly strained carbon-carbon bonds. This leaves 55 other carbon molecules in FDSP available for subsequent reaction to make the derivatives of the present invention. It is understood that FDSP may also be used as-received to function as a surfactant adjuvant to help deliver any part of the composition of the present invention.

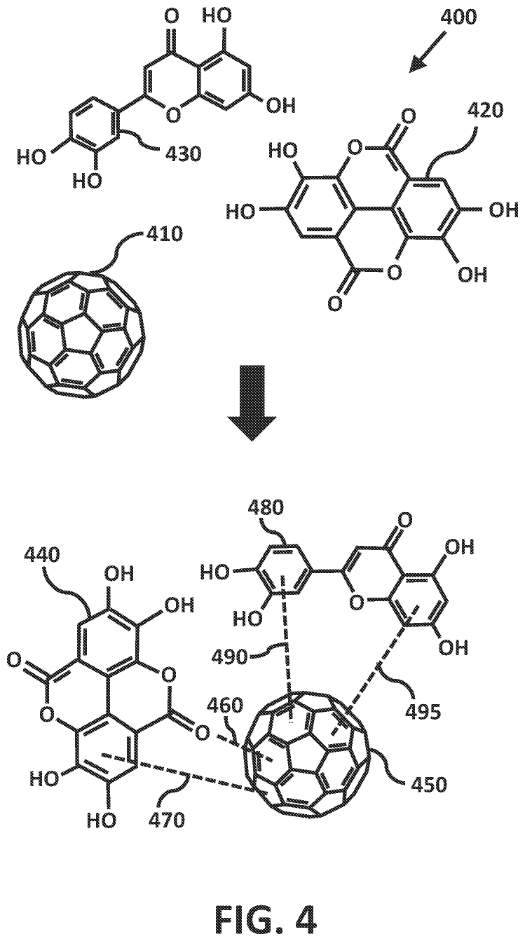

FIG. 4 illustrates the molecular structures for a chemical reaction between C60, ellagic acid and/or luteolin. The C60 reactant molecule 410 has aromatic delocalized pi electrons capable of forming pi-bonding adducts with at least one ellagic acid molecule 420 and/or at least one luteolin molecule 430. Reaction shear milling, for example, can be used to form the product indicated by the direction of the large black arrow. During reaction shear milling, an applied shear pressure of about 20 grams per square micron is sufficient to create a slightly geometric oblate spheroid of the C60 410 and simultaneously shift the density of states of the electrons of the C60 410 carbon cage into anisotropic electrostatic distributions. These induced charges then achieve a metastable state when abutted proximal to simultaneously induced opposing charges with at least one reactant polyphenolic ellagic acid 420 and/or luteolin 430. Any of the carbonyl (C═O) functional groups of at least one ellagic acid 440 in the product molecule forms a pi-carbonyl adduct with C60 450 as indicated by dashed line 460, and any of the 6-membered aromatic rings of ellagic acid 440 may form pi-pi aromatic stacking adducts as indicated by the dashed line 470. Simultaneously, at least one luteolin 480 forms adducts with C60 450 through pi-pi aromatic bonds 490, 495. It is understood that FDSP is roughly equivalent to C60 450 as a starting material, however the C60 functional group is a more effective anti-bacterial substance, and it has superior skin brightening properties to favor this variation for the method of topical skin application. Supplemental pure FDSP (not shown here) can be added to this composition to provide optional surfactant properties that stabilize the topical cream emulsion of C60-ellagic acid-luteolin, where the FDSP is to be used as an adjuvant in one method of formulation.

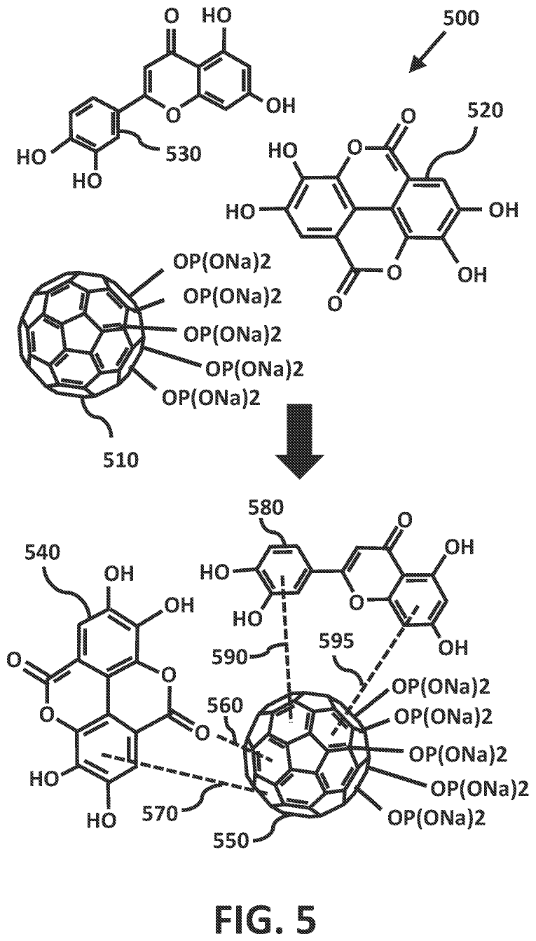

FIG. 5 illustrates a chemical reaction between C60-disodium phosphonate (FDSP), ellagic acid and/or luteolin. The FDSP reactant molecule 510 has aromatic delocalized pi electrons capable of forming pi-bonding adducts with at least one ellagic acid molecule 520 and/or at least one luteolin molecule 530. Reaction shear milling can be used, for example, to form the product indicated by the direction of the large black arrow. During reaction shear milling, an applied shear pressure of about 20 grams per square micron is sufficient to create a slightly geometric oblate spheroid of the C60 and simultaneously shift the density of states of the electrons of the fullerene carbon cage of FDSP into anisotropic electrostatic distributions. These induced charges then achieve a metastable state when abutted proximal to simultaneously induced opposing charges with at least one reactant polyphenolic ellagic acid 540 and/or luteolin 580. Any of the carbonyl (C═O) functional groups of at least one ellagic acid 540 in the product molecule forms a pi-carbonyl adduct with FDSP 550 as indicated by dashed line 560, and any of the 6-membered aromatic rings of ellagic acid 540 may form pi-pi aromatic stacking adducts as indicated by the dashed line 570. Simultaneously, at least one luteolin 580 forms adducts with FDSP 550 through pi-pi aromatic bonds 590, 595. It is understood that the reaction product FDSP-ellagic-luteolin is roughly equivalent to C60 as a starting material in place of FDSP, however the reactivity of phosphorous acid is too great to allow it to contact ellagic acid or luteolin directly in reaction with C60. Direct contact of ellagic acid or luteolin with phosphorous acid will result in the formation of unspecified and undesirable phenolic phosphonate impurities. To avoid this significant process setback, the reactive phosphonates must first be reacted with C60, after which the resultant C60-disodium phosphonates will not be able to produce such impurities towards the ellagic acid or the luteolin functional groups. It is clarified that the introduction of phosphonate groups is specified as one more functionality to the ellagic acid combination with C60, which results in a novel and unique molecular structure, C60-ellagic acid-luteolin-disodium phosphonate. This resultant nanoparticle moiety is a more effective surfactant substance to interface with a pulmonary surfactant for the method of nano-aerosol inhalants, such as to treat COPD. The phosphonate groups of the C60-ellagic acid-luteolin-disodium phosphonate nanoparticle ensemble also provides penetration ability to promote superior anti-cancer and anti-fungal properties for the nano-aerosol, orally ingested, and injected formulations. The disodium phosphonate functional groups furthermore promote substantially targeted reactivity with the sulfur compounds in tumors, fungal spores, and cancers for this variation of the composition of the present invention.

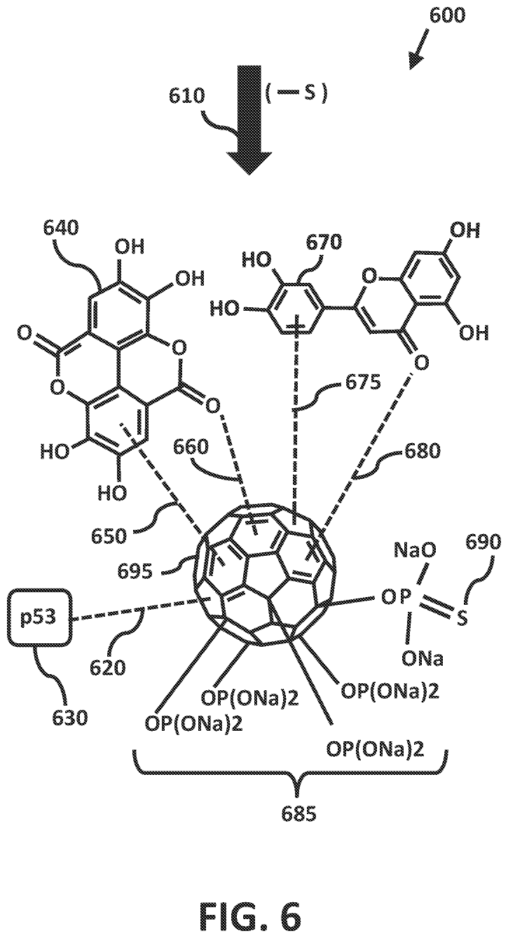

FIG. 6 illustrates a desulfurization reaction of C60-ellagic acid-luteolin-disodium phosphonate 600 of the type leading to tumor, cancer cell, and fungal spore penetration, because these structures are known to be rich in sulfur-cross-linked proteins. The direction of the desulfurization reaction for an exemplary p53 protein is illustrated by the direction of large black arrow 610. The anti-cancer and anti-tumor protective reaction is promoted by the pi-bond 620 to the core tetramer of the endogenous DNA repair protein p53 630, at the amino acid residue sequence number 91 represented herein as a schematic geometric form. The complete structure of the p53 protein 630 with all amino acid residues is available at the protein data bank under reference code 3EXJ under rcsb.org/structure/3EXJ The purpose of pi-bonding the composition of this invention to protein 630 is to allosterically hinder p53 from obtaining a ubiquitin signaling tag. Such tags are used by the human body to recycle old proteins. However, tagging this valuable protein will reduce the effective lifetime of p53. Blocking the entry of ubiquitin therefore increases the service function of this protein to repair DNA in the cell. The at least one ellagic acid functional group 640 is shown with at least one aromatic pi to aromatic pi bonds 650, and at least one aromatic pi to carbonyl bond 660. The at least one luteolin functional group 670 is shown with at least one aromatic pi to aromatic pi bonds 675, and an aromatic pi to carbonyl bond 680. As many as four of the disodium phosphonates 685 exposed to a sulfur containing compound in this case may remain in an oxidation state of 3 and are yet unreacted with sulfur. However, any one of the five disodium phosphonate groups can extract at least one sulfur atom 690 to allow the abutting phosphorous atom to arrive at a sulphurated phosphate with an oxidation state of 5, in this example of the desulfurization reaction. Desulfurization assists the human body to penetrate the crosslinked sulfur containing proteins which protect tumor and cancer cells from being detected by killer T cells and other immune responses. The source of extracted sulfur represents a local excess of glutathione and sulfur-protein bonds associated with the waxy region that separates tumor cells from the native immune system carried by aqueous phase physiological plasma such as blood in the circulatory system. Sulfurization demonstrates the superiority of FDSP-ellagic acid-luteolin over C60-Ellagic acid-Luteolin in penetrating the regions where p53 was rendered inactive by protein misfolding and entrapment by sulfur bonded protein regions.

A critical function achieved by C60-ellagic acid-luteolin further promoted by the surfactant properties of the phosphonate functional groups is to form aromatic C60 pi bonds at a region near the N-terminus of p53 and thereby block the chemical association of DM2 to the transactivation domains of p53. This region is localized to a few tyrosine and tryptophan amino acid functional group residues at a central docking location of the N-terminus (medically known to be at or about tryptophan amino acid residue number 91) by means of aromatic pi-pi bond 620 to the C60 functional group 695 thereby significantly stabilizing this complex and improving the DNA repair function of p53 beyond that expected for the unreinforced p53 protein in its natural state without a such a pi-bonded nanoparticle complex. In the case of FDSP-ellagic acid-luteolin, any of the remaining four disodium phosphonate groups 685 may continue to act as desulfurization agents, as these can provide additional subsequent desulfurization reactions, thereby enabling p53 protein complexed with an acceptable variation of the composition of the present invention to bond with p53 while the FDSP derivatives such as FDSP-ellagic acid-luteolin penetrates even more deeply into the waxy sulfurized coatings around tumor and cancer cells, according to these teachings. Supplementary information with molecular structures and amino acid residues of p53 are publicly available at the protein data bank website rcsb.org/structure/2XWR. In like manner, the DM2 bound to the transactivation domain of p53 is publicly available at rcsb.org/structure/1YCR.

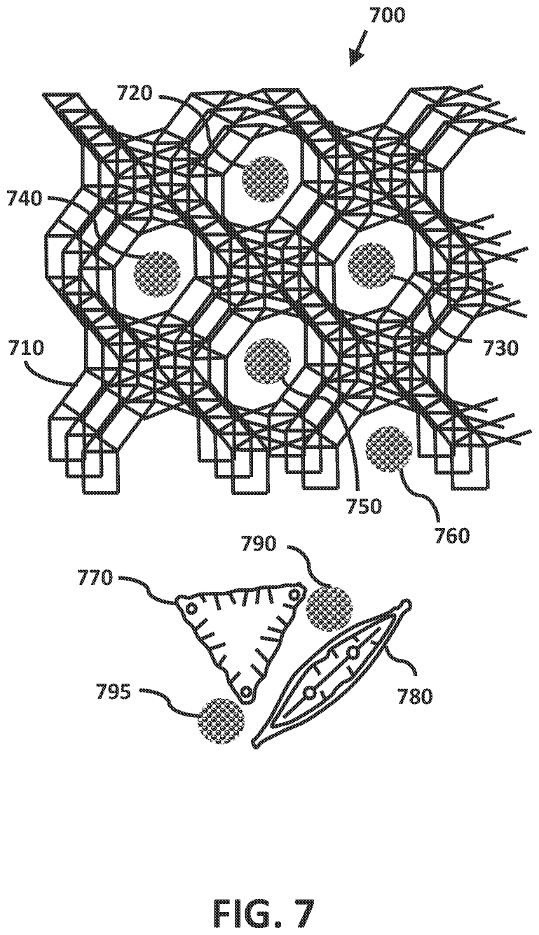

FIG. 7 illustrates the porous substrate zeolite and/or diatomaceous earth 800 impregnated with C60-ellagic acid-luteolin and/or FDSP-ellagic acid-luteolin and variations of C60 and/or FDSP having the ellagic acid derivative and/or the luteolin derivative. Transcarpathian zeolite (clinoptilolite) 710 is a type of mineral provided with a highly negative charged network structure achieving a system of reproducible and well-defined pores and galleries. Clinoptilolite zeolite 710 is well known to adsorb nitrogen containing compounds including ammonia, amino acids, and other positive charged molecules. Similarly, Clinoptilolite zeolite 710 may be optionally used herein to adsorb thiamine (vitamin B 1) as a nutritional positive counter-ion and hydrogen bonding adduct to stabilize the impregnation with the composition of the present invention. The locations of a multiplicity of pores contain the molecules of the present invention at 720, 730, 740, 750, 760. Likewise, the method of pore entrapment in solids can utilize a diatom mineral substrate 770, 780 with pore regions being greater than 100 nanometers and less than about 5 microns in size to reversibly store and later release the molecules of the present invention into the digestive tract after oral ingestion. The salt and pH moderated regenerant property of clinoptilolite and diatomaceous earths containing porous diatom particles, have reversible expression and release of molecules and compounds that has led to the widespread economic commercial adoption of clinoptilolite Transcarpathian zeolite and diatoms as a dietary supplement as well as a solid phase carrier material suitable for oral administration formulation. Other solid phase materials such as calcium phosphate and/or other solid pharmaceutical grade minerals may be used as adjuvant delivery or timed-release delivery in any combination whatsoever, to perform timed digestive release of the molecules of the composition of the present invention as methods of oral delivery, according to these teachings.

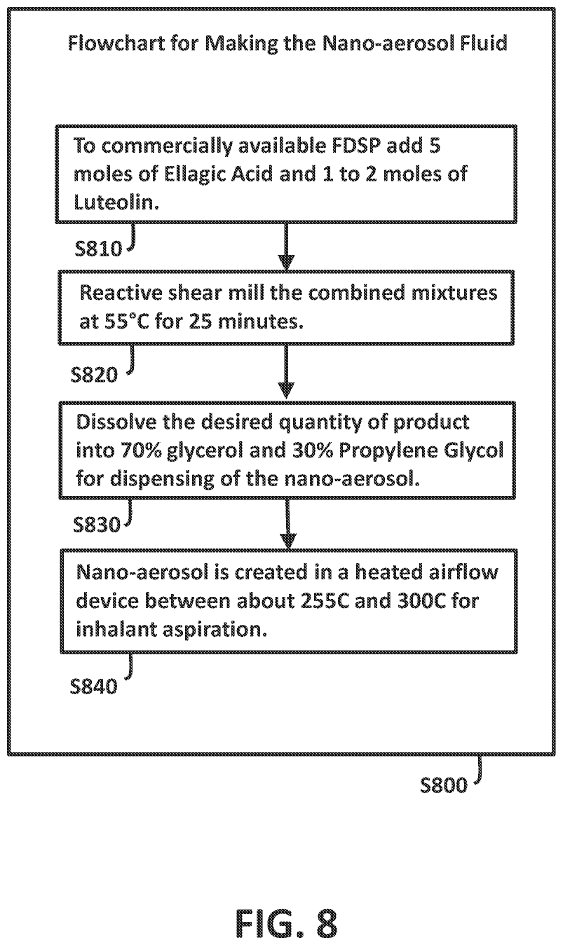

FIG. 8 is a flowchart representation of an exemplary scalable method S800 for the synthesis of nano-aerosol formulated for inhalant administration of FDSP-ellagic acid-luteolin and variations of FDSP having the ellagic acid derivative and/or the luteolin derivative. In step S810 Combine one mole of FDSP with 5 moles of ellagic acid and or 1 to 2 moles of luteolin. In step S820 the prepared dry powder mixture is reaction shear milled at about 55° C. to achieve the desired product. In this process, a shear pressure of about 20 grams per square micron is sufficient to create a slightly geometric oblate spheroid of the C60 functional group within the FDSP and simultaneously shift the density of states of the electrons of the carbon cage molecule into anisotropic electrostatic distributions to achieve a metastable state when abutted to simultaneously induced opposing electrostatic charges. The pi-bonding reactions will then occur with at least one abutting proximal ellagic acid and at least one abutting proximal luteolin molecule. In step S830, a desired concentration of product molecules is created by dissolving a weighed amount of the dry powder into a 70% glycerol and 30% polypropylene glycol solvent mixture by volume. In step S840, a metered amount of the nano aerosol fluid from step S830 is generated, for instance, by a commercially available electronic dispensing device suitable for client inhalant aspiration by means of a heated airflow between about 255° C. and 300° C. to create the nano-aerosol, according to the teachings of the present invention.

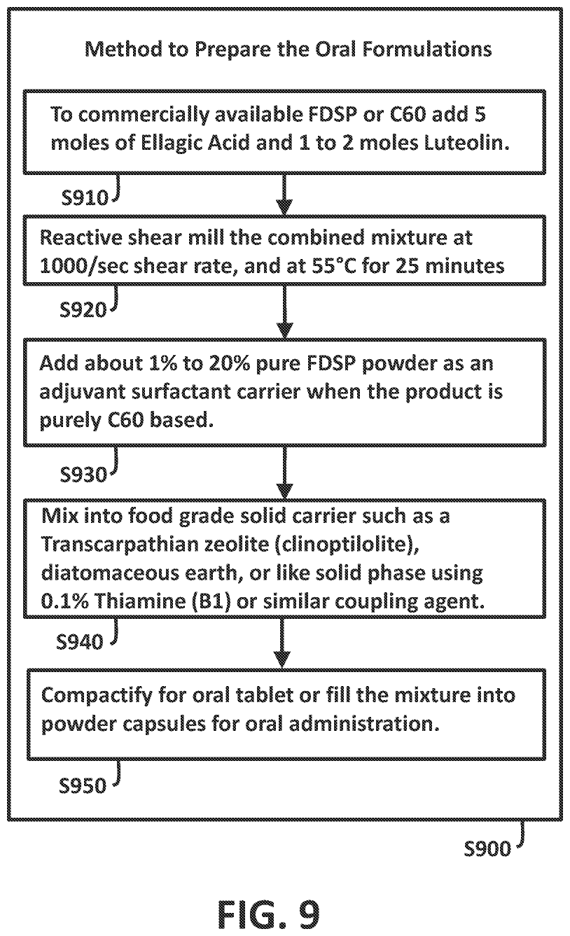

FIG. 9 is a flowchart representation of an exemplary scalable synthesis method S900 for oral administered molecules of the present invention, C60-ellagic acid-luteolin or FDSP-ellagic acid-luteolin and variations of C60 or FDSP having the ellagic acid derivative and the luteolin derivative. In step S910 Combine one mole of FDSP or 1 mole of C60, 5 moles of ellagic acid, and 1 to 2 moles luteolin. In step S920 the prepared dry powder mixture is reaction shear milled at about 55° C. to achieve the desired product. In this process, a shear pressure of about 20 grams per square micron is sufficient to create a slightly geometric oblate spheroid of the C60 or FDSP and simultaneously shift the density of states of the electrons of the carbon cage molecule into anisotropic electrostatic distributions to achieve a metastable state when abutted to simultaneously induced opposing electrostatic charges. The pi-bonding reactions then occur with at least one abutting proximal ellagic acid and/or at least one abutting proximal luteolin molecule. In step 930, about 1% to 20% pure FDSP powder is added as an adjuvant surfactant carrier when the reaction product was created with phosphonate free C60. This is to help wet and distribute the final product. In step 940, the mixture from step 930 is added into a food grade slow-release solid carrier material such as a Transcarpathian zeolite (clinoptilolite), diatomaceous earth, or a like porous solid phase. This operation can be substantially enabled by the introduction of vitamin B1 or an equivalent nitrogen containing nutrient to assist with creating a positive counter-ion charge coupling with both the negative charges in the porous solid phase and the molecules of the present invention. This process can be performed in a standard industrial kneading device such as food processing mixers that are typically used for making bread. In step S950, a desired concentration of the powder is created by dissolving a weighed amount of the dry mixture with the porous scaffold component into a mold for pressing into an oral tablet. Alternatively, capsules are filled with a weighed dosage of this final mixture for oral administration. The serving size or dosage may then be dispersed into any amount of water if desired, prior to oral administration, to ease consumption. It is understood that such a water dispersion of the time-release formulation is unstable and subject to settling on standing for periods of greater than a few hours. When desired, this formulation may be dispensed into aqueous media for later distribution at any time for later oral administration, with the provision of optional viscosity modifiers that are added to this mixture to stabilize the insoluble mineral components from settling therein as a minor variation to this method. This enhances the long-term esthetic appeal of the solid dispersed formulated product into aqueous media, while simultaneously maintaining the time-release feature of the porous solid insoluble carriers, according to the teachings of the present invention.

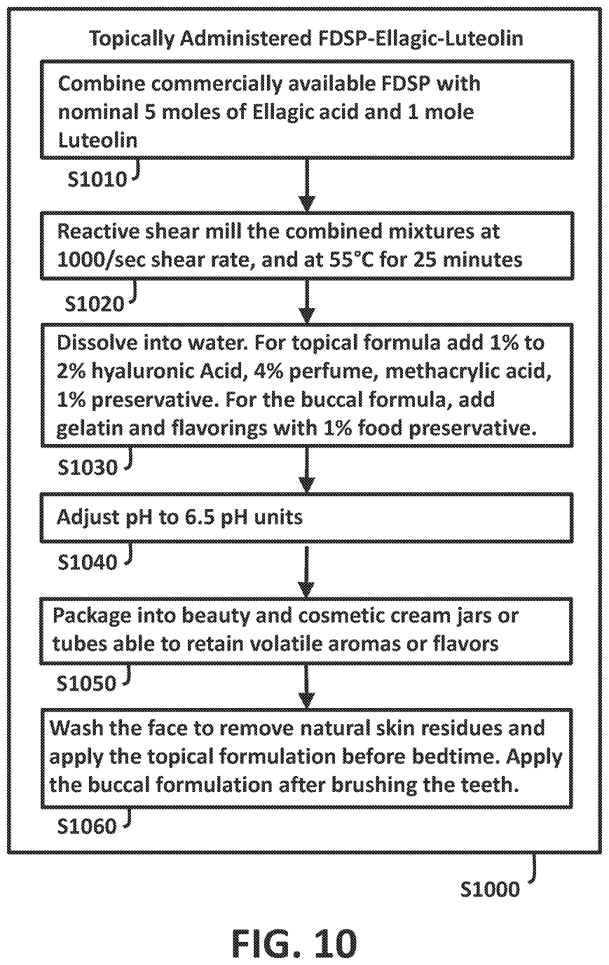

FIG. 10 is a flowchart representation of a synthesis S1000 of topical skin or buccal administered molecules of the present invention, C60-ellagic acid-luteolin and/or FDSP-ellagic acid-luteolin and variations of C60 and/or FDSP having the ellagic acid derivative and/or the luteolin derivative. In step S1010 One mole of FDSP is combined with nominal 5 moles of Ellagic acid and 1 mole Luteolin. (Note: C60 raw material is not preferred for these formulations). The range of these components may be altered somewhat depending on efficacy requirements and may be limited by steric availability to react beyond the compositions reported herein. In step S1020 the mixture of step S1010 is reaction shear milled at about 55° C. to achieve the desired molecular reaction product. In step S1030, the reaction product is dissolved into water. For topical skin formulation, 1% to 2% hyaluronic acid, about 4% perfume, a desired amount of methacrylic acid for viscosity enhancement, and 1% preservative are added. However, for buccal solutions, gelatin and desired flavors with 1% sodium sorbate as a food preservative can be used. In step S1040, the pH of the composition is adjusted to prevent mold or bacterial growth with an acceptable range of 5 to 6.7 with a nominal value of 6.5 pH by neutralization with sodium hydroxide (NaOH) with adequate mixing to ensure a uniform cream or lotion. In step S1050, the material composition is transferred into beauty and cosmetic cream jars or tubes having a sufficiently hermetic seal to able to retain the volatile aromas or flavorings. In step 1060, the face is washed to remove natural skin residues before applying the topical formulation, such as before bedtime. The buccal formulation can be applied after brushing teeth and rinsing, for example.

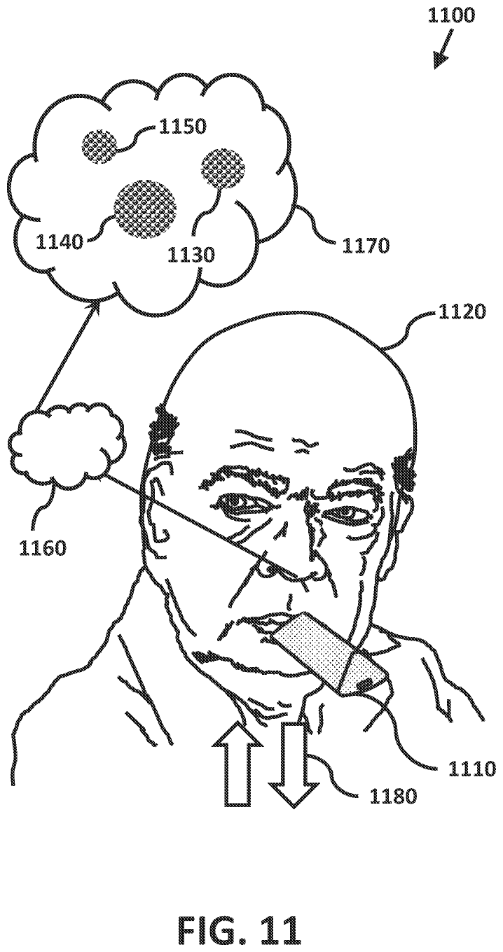

FIG. 11 illustrates personal administration 1100 of an aspirated nano-aerosol delivery solution containing the molecules of the present invention. A nano-aerosol generating device 1110 filled with the fluid mixture containing the molecules of the present invention as an inhalant dispensing solution is provided for dispersing the created inhalant gas wherein the nano-particles are nebulized. The dispensing device 1110 may also be more commonly known as a nebulizer, or an electronic vaporizing device, or an electronic cigarette, or the functional part of a hookah to be shared among several users. In all cases these systems serve to carry the composition in a carrier fluid dispenser 1110, move that composition in nebulized form along with an aerosolized solvent, and transfer this composition into a substantially gaseous dispersion directed into the nose, mouth, trachea, and airways of a patient or user 1120. One intended use of the composition is to treat, delay or arrest the incidence of cancers wherein the nano-aerosol can expedite targeted delivery to the brain by avoiding a passage through the digestive system. Another intended use is to treat COPD using the reacted FDSP composition of the present invention.

Some of the nano-aerosolized composition is exhaled and shown as particulate clusters 1130, 1140, 1150 within exhaled smoke puffs 1160 and 1170 emitted on exhalation as indicated by the direction of thin line arrows radiating away from the nose of the subject 1120. Delivery of the nano-aerosol composition from dispenser 1110 provides antioxidant properties to the mucus airway tissues wherein destruction of free radicals and oxidants associated with cancer (especially lung cancer), or valley fever, or are used to treat COPD as provided using this method. Systems that may be used for the method of dispersion of the nano-aerosol fluid is represented by dispenser 1110, and include, without limitation, any of the electronic cigarette devices produced internationally and listed in Appendix 4.1, “Major E-cigarette Manufacturers” of the “2016 Surgeon General's Report: E-Cigarette Use Among Youth and Young Adults” published by the Center for Disease Control and Prevention (CDC), Office of Smoking and Health (OSH) freely available at the CDC.GOV website, and/or any combination of piezoelectric, resistively heated, or inductively heated vaporized fluid delivery methods that can be utilized to deliver the composition of the present invention, especially when such a device is approved as a medical drug delivery device. Each embodied variation of such methods without limit are intended to aspirate aerosols as the method of therapeutic substance delivery of the composition of the present invention directed into the nasal cavities, mouth, tracheal breathing orifice, or intubated trachea of a patient. The supply direction of nebulized feed on inhalation and exhalation are delivered into the airways and lungs of the intended patient by the flow of supplied air as indicated by the direction of upward and downward facing large white arrows 1180, when used according to the teachings of the present invention.

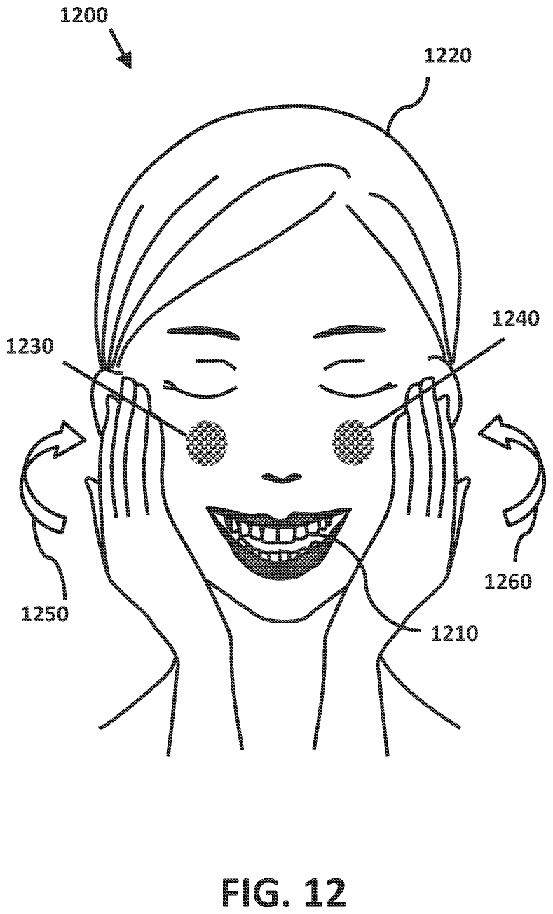

FIG. 12 illustrates a personal topical skin administration and/or buccal administration method 1200 of the composition of the present invention, C60-ellagic acid-luteolin and/or FDSP-ellagic acid-luteolin and variations of C60 and/or FDSP having the ellagic acid derivative and/or the luteolin derivative. A semi-liquid slurry dispersion, cream, ointment, or lotion can be used to contain and transfer the administered formula, as a somewhat different formulation is required depending on whether the application is for topical use on the skin or to coat tooth surfaces in the oral or buccal cavity, which is located inside the mouth. 1210. The skin care formula can be applied by the user 1220 in regions such as the face 1230, 1240. Application of the skin care formulation can be by means of circular rubbing motions as indicated by the direction of arrows 1250, 1260. The skin-care formulation then confers topical antimicrobial, anti-aging and skin brightening functions, and promotes resistance to the onset of skin cancers. In the case of the buccal administration to the oral cavity 1210, the oral mucosal antibacterial functions promote anti-gingivitis properties, as well as anti-esophageal cancer treatment properties. The formulations are to be synthesized and administered according to the teachings of the present invention.

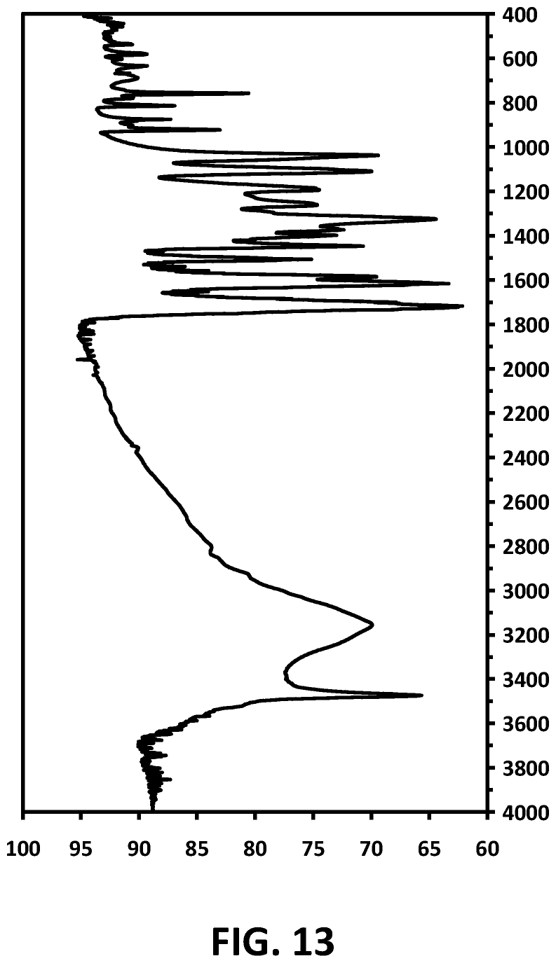

FIG. 13 illustrates experimental FTIR data for ellagic acid raw material. This sample and all subsequent samples tested by FTIR herein were prepared by the method of mixing, crushing, and consolidating under 7 metric tons of pressure, about 0.001 grams of analyte with 1 gram of a diluent solid material that is substantially transparent to infrared light, this diluent being anhydrous potassium bromide (KBr), which then flows under pressure to form a translucent pellet of about 0.4 mm thickness. Spectral background subtraction in air using a control pellet of the same mass and thickness having pure KBr was used to obtain a baseline instrument infrared spectral transmission response. This method is generally referred to as the ‘KBr pellet’ sample preparation method, and it is used hereinafter throughout for each FTIR experimental data collection and spectral analysis. The Fourier transform infrared spectrophotometer used herein to obtain FTIR spectra throughout, is a model RF6000 FTIR instrument manufactured by Shimadzu of Japan.

The sample of ellagic acid analyte prepared by KBr pellet includes a narrow and sharp absorbance peak at 3473 reciprocal centimeters (hereinafter cm⁻¹) that arises from anhydrous (—OH) hydroxyl groups, in which this absorbance disappears in the monohydrate form, whereas the 3155 cm⁻¹ absorbance peak arises from the bulk (—OH) hydroxyl bending. The absorbance at 1506 cm⁻¹ arises from typical aromatic resonance delocalized carbon to carbon bonds (C═C) from benzene ring structures. The absorbance at 1039 cm⁻¹ is characteristic of (C—O) carbon oxygen stretch. The narrow and sharp absorbance at 1717 cm⁻¹ arises from (C═O) stretching band of the carbonyl functional groups. The broad triangular absorbance at 3153 cm⁻¹ arises from the stretching band of the four acidic hydroxyl groups that are pendant from the benzene ring portion of the ellagic acid molecular structure. The overall infrared spectrum test data agrees with the published public spectra for ellagic acid.

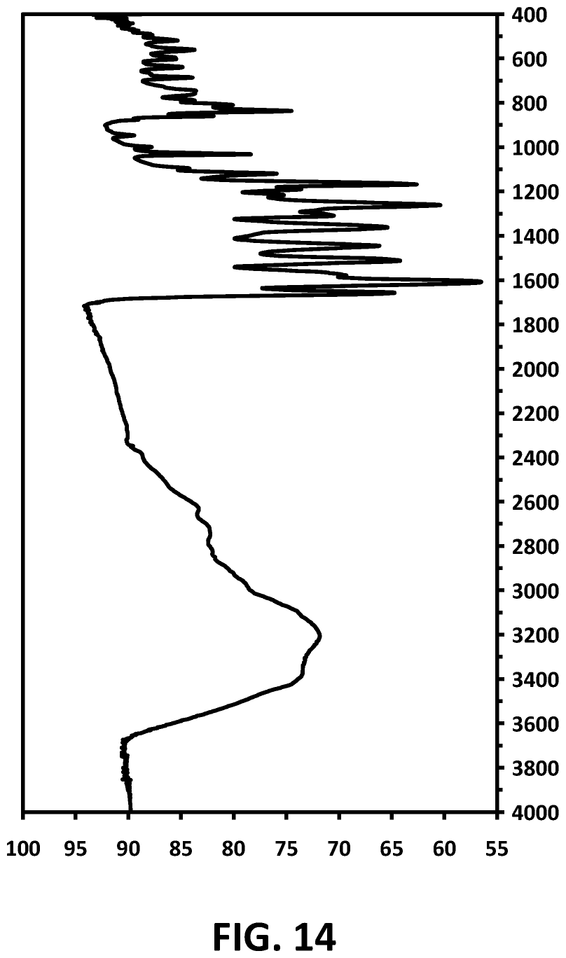

FIG. 14 illustrates experimental FTIR data for luteolin bioflavonoid. The absorbance at 3186 cm⁻¹ represents hydroxyl group bending. The characteristic carbonyl stretching absorbance band for luteolin is observed at 1656 cm⁻¹. The absorbance at 1363 cm⁻¹ is from the ortho phenolic (—C—OH) hydroxyl group stretching. The band at 1168 cm⁻¹ arises from the (C—O—C) oxygen carbon ring stretching. The overall infrared absorbance spectral features are consistent with and indicate chemical similarity to the published public spectra for luteolin bioflavonoid.

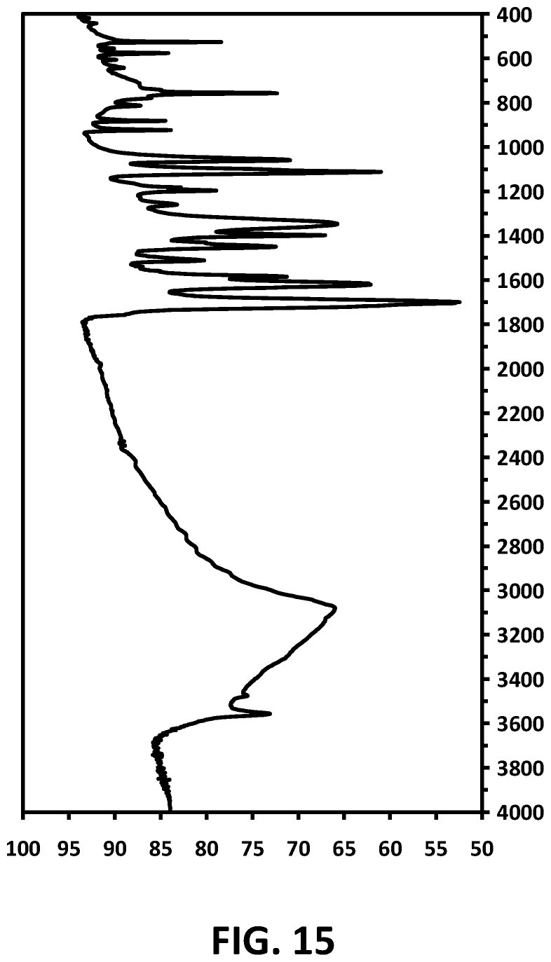

FIG. 15 illustrates experimental FTIR data for C60-ellagic acid-luteolin. The very sharp and narrow absorbances at 526 cm⁻¹ and 576 cm⁻¹ are characteristic for the C60 fullerene group. By comparison with FIG. 13 for ellagic acid, the previous narrow and sharp absorbance at 1717 cm⁻¹ from the (C═O) stretching band of the carbonyl functional groups of ellagic acid have shifted to 1700 cm⁻¹, indicating a significant change in their chemical environment that is attributed to their pi-carbonyl bonding with the C60 aromatic regions. The broad absorbance at 3077 cm⁻¹ arises from the phenolic acidic hydroxyl groups (—OH) pendant from both the ellagic acid and the luteolin bioflavonoid. The carbonyl (C═O) absorbance at 1620 cm⁻¹ does not appear in either luteolin or ellagic acid, therefore indicating either or both compounds have another functional type of altered carbonyl expression, likely again because of a significant change in their chemical environment that is attributed to the pi-carbonyl bonding with the C60 aromatic regions. The characteristic benzene ring carbon to carbon bond stretching is still present at 1506 cm⁻¹, however the one at 1463 cm⁻¹ appears to have shifted to 1448 cm⁻¹, indicating a significant structural resonance mode change for the C60 delocalized cage molecule with those aromatic regions of the new functional groups having significant pi-pi stacking interactions.

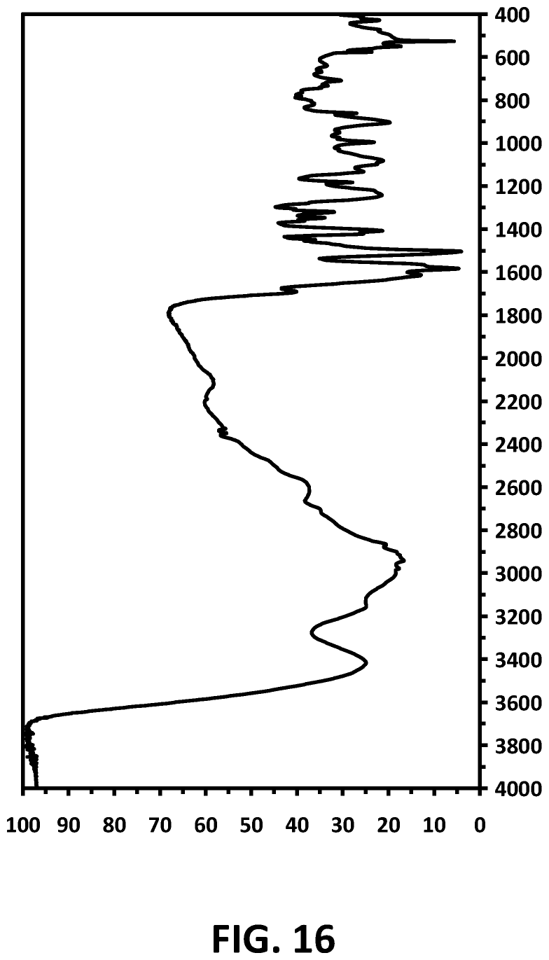

FIG. 16 illustrates the experimental FTIR data for FDSP. The absorbance at 3410 cm⁻¹ arises from phosphorous pendant hydroxyl groups (P—OH) at those locations where the sodium atom was replaced by a hydrogen atom. Absorbances at 1508 cm⁻¹ and 1586 cm⁻¹ are characteristic of resonance delocalized fullerene carbon atoms (C═C). The weak broad peaks at 580 cm⁻¹ to 561 cm⁻¹ indicate a characteristic phosphonate absorbance. The very sharp and narrow absorbances at 526 cm⁻¹ and 576 cm⁻¹ are characteristic for the C60 fullerene group. The overall infrared absorbance spectral features are consistent with the typical FTIR spectrum for commercially available FDSP.

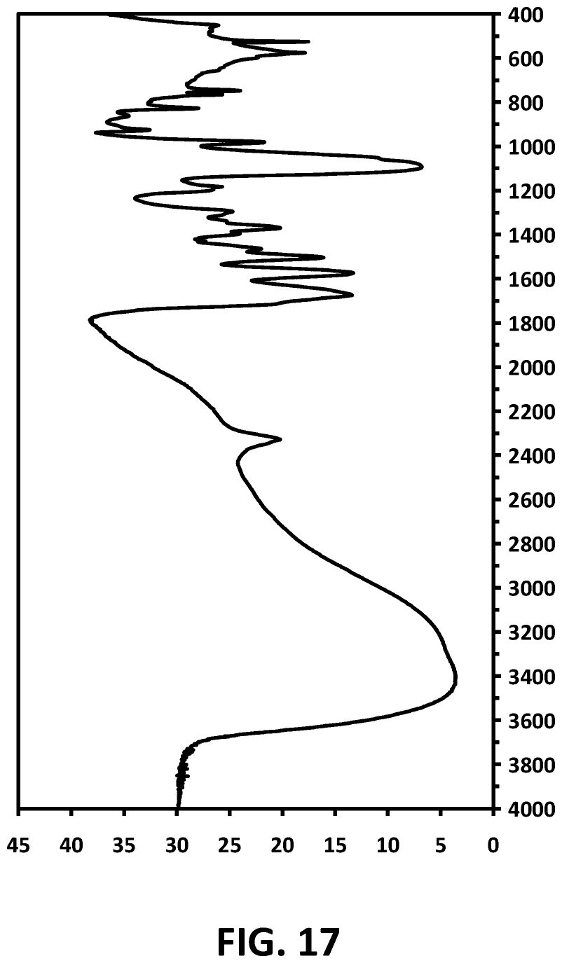

FIG. 17 illustrates experimental FTIR data for FDSP-ellagic acid-luteolin. By comparison with FIG. 13 for ellagic acid, the previous narrow and sharp absorbance at 1717 cm⁻¹ from the (C═O) stretching band of the carbonyl functional groups of ellagic acid have completely vanished and have been replaced by a new carbonyl absorbance at 1672 cm⁻¹. This finding shows that pi-carbonyl bonds between ellagic acid and the fullerene C60 core molecule had formed, and the structure of these complexes was very dense. The characteristic benzene ring carbon to carbon bond stretching is still present at 1505 cm⁻¹ and 1463 cm⁻¹. A significant new and very strong absorbance at 1088 cm⁻¹ is attributed to the pi-cation interaction of the sodium phosphonate groups pendant from the fullerene with the aromatic benzene structures of ellagic acid. The broad absorbance at 3396 cm⁻¹ arises from the phenolic acidic hydroxyl groups (—OH) pendant from luteolin bioflavonoid, whereas the shoulder at 3186 cm⁻¹ was contributed by the conserved hydroxyl groups (—OH) pendant from ellagic acid. The absorbance at 526 cm⁻¹ is also seen for phosphorous acid, indicating there was a successful transfer of the phosphonate group (P—OH) to the fullerene, and it remained a viable functional group even after the full reaction with ellagic acid and luteolin. Overall, the intensities of infrared absorbances show an extensive redistribution of vibrational intensities associated with conformational and structural bonding changes that are caused by interactions with the fullerene molecular environment. These changes do not stay localized in a narrow part of the spectrum but affect and uniquely characterize practically the whole spectrum for the FDSP ellagic acid luteolin composition.

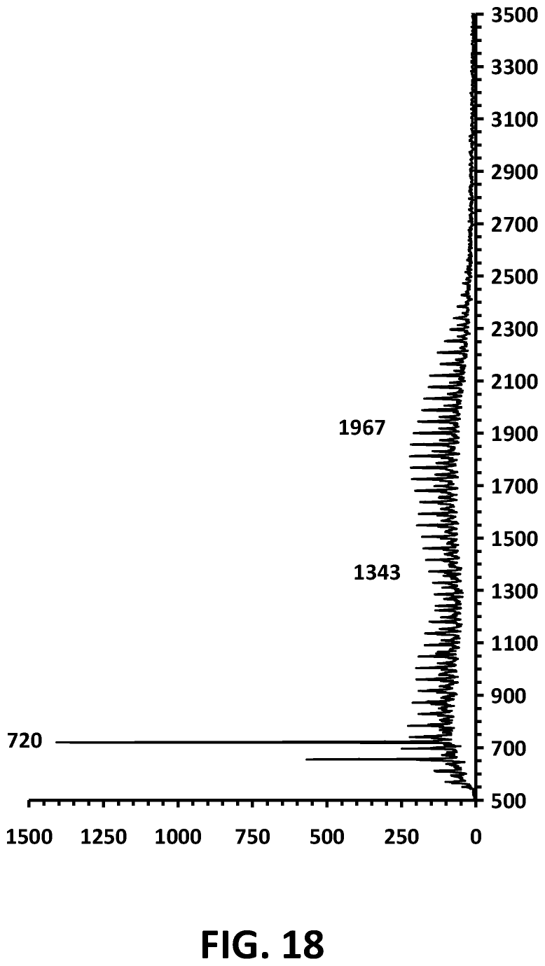

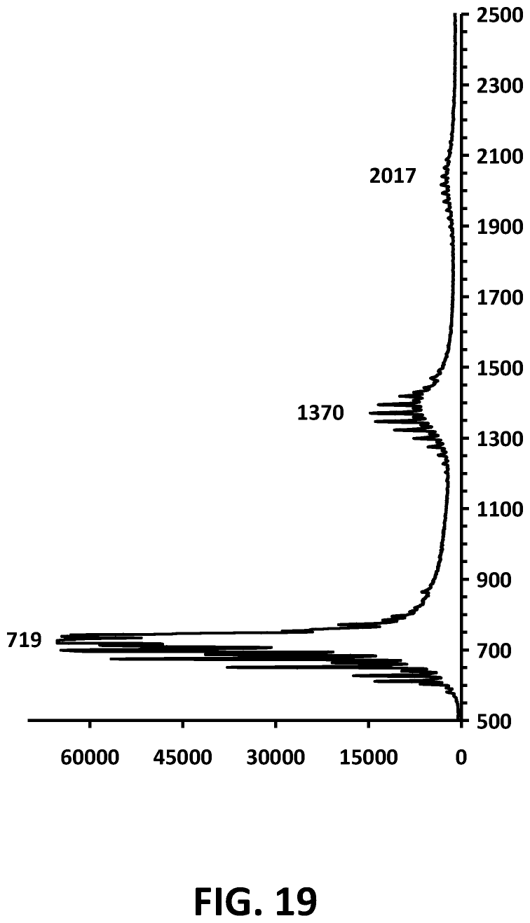

FIG. 18 illustrates experimental negative mode MALDI-TOF mass spectrograph data for FDSP raw material. This test sample and each of those that follow herein resulted from being diluted with water, after which the aqueous sample was then introduced in a vaporized state into a Voyager Mass Spectrograph from Applied Biosystems (Foster City, Calif., USA). Negative mode bombardment was by fast moving electrons at about 70 eV energy. This resulted in ion formation. One electron from the highest orbital energy was dislodged, and therefore, molecular ions were formed. Some of these molecular ions underwent spallation, and the subsequent fragment ions were formed. The fragmentation of the ions was because of the excess of applied energy that it acquired within the ionization chamber; only negative ions were recorded. The largest peak observed was the primary and core molecular ion, this being a fullerene ion as indicated by the numeric peak label at mass to charge ratio of 720. The primary molecular ion was subsequently verified using a pristine pure reference material of C60 tested immediately after this test, under both negative mode and positive mode test conditions (the check standard results are not shown here). The observed mass chromatographic spallation ions greater than the primary molecular ion, formed peaks that were separated into two distinctly charged groups with their respective peak masses clustered about local maxima at 1343 and 1967 mass to charge ratios (m/z), and were respectively assigned to a nominal C60(O₃PNa₂)₅ composition, and a trace nominal C60(O₃PNa₂)₁₀ composition based on a combinatorial ionic mass and charge analysis. FIG. 19 illustrates experimental negative mode MALDI-TOF mass spectrograph data for the FDSP-ellagic acid-luteolin composition. The largest peak observed was the primary and core molecular ion, this being a fullerene ion as indicated by the mass to charge ratio of 719. The mass to charge ratio of 1370 is attributed to five groups of disodium phosphonate pendant from fullerene C60 as FDSP. The heaviest mass to charge peak of about 2017 is consistent with FDSP having two pi-carbonyl bonded ellagic acid groups.

As variations, combinations and modifications may be made in the construction and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments but defined in accordance with the foregoing claims appended hereto and their equivalents.

What is claimed is:

1. A nanoparticle composition comprising:
buckminsterfullerene (C60) bonded to ellagic acid.

2. The nanoparticle composition of claim 1 wherein a bond between the C60 and the ellagic acid is a pi bond.

3. The nanoparticle composition of claim 1 wherein the C60 is further bonded to luteolin.

4. The nanoparticle composition of claim 3 wherein a bond between the C60 and the luteolin is a pi bond.

5. The nanoparticle composition of claim 1 wherein the C60 is further bonded to a disodium phosphonate functional group.

6. The nanoparticle composition of claim 5 wherein the C60 is bonded to the disodium phosphonate functional group and further bonded to four additional phosphonate functional groups.

7. The nanoparticle composition of claim 1 further comprising a zeolite, wherein the C60 bonded to ellagic acid is disposed within the zeolite.

8. The nanoparticle composition of claim 1 further comprising diatomaceous earth, wherein the C60 bonded to ellagic acid is disposed within porous diatom particles of the diatomaceous earth.

9. The nanoparticle composition of claim 1 further comprising a solvent, wherein the C60 bonded to ellagic acid is disposed in the solvent.

10. The nanoparticle composition of claim 9 wherein the solvent comprises a mixture of 70% glycerol and 30% polypropylene glycol by volume.

11. A method of curing, treating, or prophylactically avoiding cancer, valley fever, or COPD, in a subject, comprising the step of:
administering to the subject an effective amount of a composition including a buckminsterfullerene (C60) bonded to ellagic acid.

12. The method of claim 11 wherein the composition includes a pharmaceutically acceptable carrier and the C60 bonded to the ellagic acid is disposed in the pharmaceutically acceptable carrier.

13. The method of claim 12 wherein the pharmaceutically acceptable carrier comprises a zeolite or diatomaceous earth.

14. The method of claim 12 wherein the composition disposed in the pharmaceutically acceptable carrier comprises a tablet, capsule, pill, powder, granule, or a liquid.

15. The method of claim 11 wherein administering the composition comprises administration by an intravenous, intramuscular, subcutaneous, intrathecal, intraperitoneal, topical, nasal, or oral route.

16. The method of claim 11 wherein administering the composition comprises administering an oral dosage including up to about 500 mg of the C60 bonded to the ellagic acid.

17. The method of claim 11 wherein administering the composition comprises administering an intramuscular, intravenous, or a subcutaneous dose of the C60 bonded to the ellagic acid in an amount of from about 0.1 mg/Kg to about 5 mg/Kg.

18. The method of claim 11 wherein administering the composition comprises administering a nano aerosol, a vapor, a powder, a dust, or an aerosolized inhalant.

19. The method of claim 11 wherein the C60 is further bonded to luteolin.

20. The method of claim 11 wherein the C60 is further bonded to a disodium phosphonate functional group.

21. A method of making a nanoparticle comprising a buckminsterfullerene (C60) bonded to ellagic acid, the method comprising:
bonding the ellagic acid to the C60.

22. The method of claim 21 wherein bonding the ellagic acid to the C60 is performed by reaction shear mixing.

23. The method of claim 21 further comprising bonding the C60 to luteolin by reaction shear mixing.

24. A method of making a nanoparticle comprising:
bonding ellagic acid to C60 disodium phosphonate to produce ellagic acid C60 disodium phosphonate.

25. The method of claim 24 wherein bonding the ellagic acid to the C60 disodium phosphonate is performed by reaction shear mixing.

26. The method of claim 24 further comprising bonding the ellagic acid C60 disodium phosphonate to luteolin by reaction shear mixing.
